(19) United States
(12) Patent Application Publication
(10) Pub. No.: US 2025/0143797 A1
(43) Pub. Date: May 8, 2025

(54) AUGMENTED REALITY REGISTRATION DEVICE FOR NAVIGATED SURGERY

(71) Applicants: Smith & Nephew, Inc., Memphis, TN (US); Smith & Nephew Asia Pacific Pte. Limited, SG (SG); Smith & Nephew Orthopaedics AG, Zug (CH)

(72) Inventors: Samuel C. DUMPE, Sewickley, PA (US); John ESTRADA, Pittsburgh, PA (US); Constantinos NIKOU, Monroeville, PA (US)

(21) Appl. No.: 18/935,851

(22) Filed: Nov. 4, 2024

Related U.S. Application Data
(60) Provisional application No. 63/595,881, filed on Nov. 3, 2023.

(51) Int. Cl. A61B 34/10; A61B 34/20; A61B 90/00; A61B 90/50

(52) U.S. Cl. CPC A61B 34/10; A61B 34/20; A61B 90/361; A61B 90/39; A61B 90/50; A61B 2034/105; A61B 2034/2057; A61B 2034/2065; A61B 2090/363; A61B 2090/365; A61B 2090/502

(57) ABSTRACT

Systems and methods for performing computer-aided surgical navigation using augmented reality are disclosed. An augmented reality marker assembly including a tracking marker configured for use with an augmented reality system is configured to be attached to a tracking device at a fixed orientation. The tracking marker includes a contrasting three-dimensional surface. The system can sense and determine a three-dimensional orientation of the augmented reality marker and the tracking device in their respective reference frames.

### CROSS-REFERENCE TO RELATED APPLICATION

This application claims the benefit of priority to U.S. Provisional Application No. 63/595,881 titled “AUGMENTED REALITY REGISTRATION DEVICE FOR NAVIGATED SURGERY” filed Nov. 3, 2023, which is incorporated herein by reference in its entirety.

### TECHNICAL FIELD

The present disclosure relates generally to methods, systems, and apparatuses related to a computer-assisted surgical system that includes various hardware and software components that work together to enhance surgical workflows. The disclosed techniques may be applied to, for example, shoulder, hip, and knee arthroplasties, as well as other surgical interventions such as arthroscopic procedures, spinal procedures, maxillofacial procedures, rotator cuff procedures, ligament repair and replacement procedures.

### BACKGROUND

Augmented reality (AR) is an interactive experience by which objects that reside in the real world are “augmented” by computer-generated information. Augmenting information can be constructive (i.e., additive to the natural environment) or destructive (i.e., masking of the natural environment) and may be interwoven with the physical world such that a viewer perceives the information as an immersive aspect of the real environment. This augmentation can function across one or more sensory modalities, including visual, auditory, haptic, somatosensory, and olfactory senses.

A near-eye display system may be used for augmented reality. With such a system, a scene that is being viewed by a user of the assembly can be altered. For example, the scene may be augmented or supplemented with visual material. The alteration may be computer-generated and include real-time video and/or non-real-time images that are presented to a user while gazing at the scene.

Utilizing augmented reality in navigated surgery requires consistent registration between the navigation system and the augmented reality system. Typically, each system has its own unique global coordinate system, and a method is required to communicate the relative position of the two global coordinate systems so that data acquired from the navigation system can be displayed accurately in the AR display.

A system is needed that implements a co-registered AR video display and a surgical navigation system intraoperatively. Furthermore, the system should integrate into existing systems with existing trackers and separate AR video cameras and surgical navigation sensors. U.S. patent application Ser. No. 17/435,845, entitled “Co-registration for augmented reality and surgical navigation”, discloses a two-dimensional tracker assembly for co-registration and is hereby incorporated herein by reference.

### SUMMARY

In some embodiments, a surgical system includes a tracking device comprising a first tracking marker configured for use with a surgical navigation tracking system, the tracking device configured to be mounted to a patient. The system further includes an augmented reality marker assembly comprising a second tracking marker configured for use with an augmented reality system, the augmented reality marker assembly configured to be attached to the tracking device at a fixed orientation, and the second tracking marker including a contrasting three-dimensional surface. The system further includes a sensor assembly configured to identify locations of each of the first tracking marker and the second tracking marker. A control system communicably connected to the sensor assembly is configured to determine a first three-dimensional orientation of the patient in a first reference frame by locating the first tracking marker via the sensor assembly, and determine a second three-dimensional orientation of the augmented reality marker assembly in a second reference frame by locating the second tracking marker via the sensor assembly.

In some embodiments, the control system is further configured to determine a transformation between the first reference frame and the second reference frame.

In some embodiments, the control system includes a display screen, wherein the control system is further configured to cause the display screen to display surgical data based on the transformation.

In some embodiments, the display screen includes at least one of a near-eye display or a head-mounted display.

In some embodiments, the control system is further configured to generate a surgical plan and wherein at least a portion of the surgical data displayed via the display screen includes the surgical plan.

In some embodiments, the contrasting three-dimensional surface includes a plurality of contrasting colors.

In some embodiments, the contrasting three-dimensional surface further includes a plurality of polygonal surfaces, wherein a color of each polygonal surface is selected from the plurality of contrasting colors.

In some embodiments, the contrasting three-dimensional surface includes one or more openings into a hollow portion of the second tracking marker.

In some embodiments, the augmented reality marker assembly includes a plurality of fastening mechanisms, and the plurality of fastening mechanisms are configured to be attached to a frame of the tracking device in a particular manner that causes the augmented reality marker assembly to assume the fixed orientation.

In some embodiments, the system further includes a plurality of tracking devices and a plurality of augmented reality marker assemblies each comprising an augmented reality tracking marker, wherein each augmented reality tracking marker is distinguishable by the sensor assembly.

In some embodiments, the sensor assembly includes a first sensor configured to identify a first location of the first tracking marker and a second sensor configured to identify a second location of the second tracking marker.

In some embodiments, the sensor assembly includes a plurality of tracking cameras.

In some embodiments, a tracking device attachment for correlating a tracking system coordinate frame with an augmented reality system coordinate frame includes a tracking marker including a contrasting three-dimensional surface configured to be identified by an augmented reality system and a plurality of fastening mechanisms configured to attach the tracking device attachment to a tracking device at a fixed orientation, wherein the tracking device comprises a marker configured to be identified by a surgical navigation system and configured to be mounted to a patient.

In some embodiments, the plurality of fastening mechanisms are configured to be attached to a frame of the tracking device in a particular manner that causes the tracking device attachment to assume the fixed orientation.

In some embodiments, the contrasting three-dimensional surface includes a plurality of contrasting colors.

In some embodiments, the contrasting three-dimensional surface further includes a plurality of polygonal surfaces, wherein a color of each polygonal surface is selected from the plurality of contrasting colors.

In some embodiments, the plurality of contrasting colors form a pattern on the contrasting three-dimensional surface.

In some embodiments, the contrasting three-dimensional surface comprises one or more openings into a hollow portion of the tracking marker.

In some embodiments, a surgical system includes a sensor assembly configured to identify a first location of a first tracking marker associated with a tracking device, the tracking device configured to be mounted to a patient, wherein the first tracking marker is configured for use with a surgical navigation system, and identify a second location of a second tracking marker associated with an augmented reality marker assembly, the augmented reality marker assembly configured to be attached to the tracking device at a fixed orientation, wherein the second tracking marker comprises a contrasting three-dimensional surface configured for use with an augmented reality system. The system further includes a control system communicably connected to the sensor assembly, the control system configured to determine a first three-dimensional orientation of the patient in a first reference frame by locating the first tracking marker via the sensor assembly and determine a second three-dimensional orientation of the augmented reality marker assembly in a second reference frame by locating the second tracking marker via the sensor assembly.

In some embodiments, the control system is further configured to determine a transformation between the first reference frame and the second reference frame.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an operating theatre including an illustrative computer-assisted surgical system (CASS) in accordance with an embodiment.

FIG. 2A depicts illustrative control instructions that a surgical computer provides to other components of a CASS in accordance with an embodiment.

FIG. 2B depicts illustrative control instructions that components of a CASS provide to a surgical computer in accordance with an embodiment.

FIG. 2C depicts an illustrative implementation in which a surgical computer is connected to a surgical data server via a network in accordance with an embodiment.

FIG. 3 depicts an illustrative tracking device in accordance with an embodiment.

FIGS. 4A and 4B depict augmented reality marker assembly integrated with a tracking device in accordance with an embodiment.

FIG. 5 is a block diagram depicting an illustrative system for providing navigation and control to a computer-assisted surgical planning system.

FIG. 6 illustrates a block diagram of an exemplary data processing system in which embodiments are implemented.

### DETAILED DESCRIPTION

This disclosure is not limited to the particular systems, devices and methods described, as these may vary. The terminology used in the description is for the purpose of describing the particular versions or embodiments only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

### Definitions

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patients worn or damaged acetabulum. While the term “implant” is generally considered to denote a man-made structure (as contrasted with a transplant), for the purposes of this specification an implant can include a biological tissue or material transplanted to replace or enhance a biological structure.

For the purposes of this disclosure, the term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

For the purposes of this disclosure, the terms “distract,” “distracting,” or “distraction” are used to refer to displacement of a first point with respect to a second point. For example, the first point and the second point may correspond to surfaces of a joint. In some embodiments herein, a joint may be distracted, i.e., portions of the joint may be separated and/or moved with respect to one another to place the joint under tension. In some embodiments, a first portion of the joint be a surface of a femur and a second portion of the joint may be a surface of a tibia such that separation occurs between the bones of the joint. In additional embodiments, a first portion of the joint may be a first portion of a tibial implant component or a tibial trial implant and a second portion of the joint may be a second portion of the tibial implant component or the tibial trial implant that is movable with respect to the first portion (e.g., a base plate and a superior spacer as described herein). Accordingly, separation may occur between the portions of the tibial implant component or the tibial trial implant (i.e., intra-implant separation). Throughout the disclosure herein, the described embodiments may be collectively referred to as distraction of the joint.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly demarcated. For example, a reference to a surgeon also could apply, in some embodiments to a technician or nurse.

The systems, methods, and devices disclosed herein are particularly well adapted for surgical procedures that utilize surgical navigation systems, such as the CORI® surgical navigation system. CORI is a registered trademark of SMITH & NEPHEW, INC. of Memphis, TN.

### CASS Ecosystem Overview

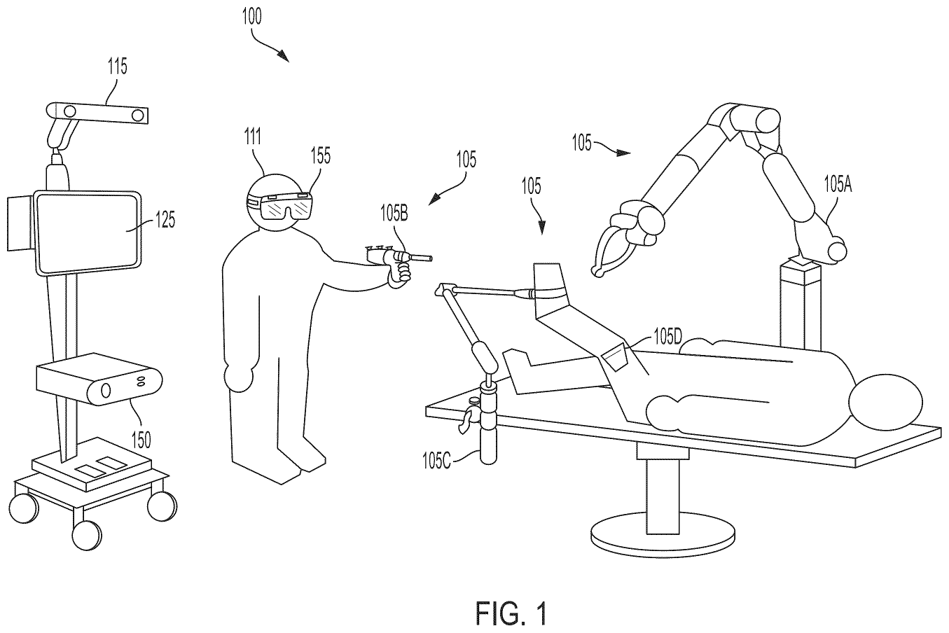

FIG. 1 provides an illustration of an example computer-assisted surgical system (CASS) 100, according to some embodiments. As described in further detail in the sections that follow, the CASS uses computers, robotics, and imaging technology to aid surgeons in performing orthopedic surgery procedures such as total knee arthroplasty (TKA) or THA. For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An Effector Platform 105 positions surgical tools relative to a patient during surgery. The exact components of the Effector Platform 105 will vary, depending on the embodiment employed. For example, for a knee surgery, the Effector Platform 105 may include an End Effector 105B that holds surgical tools or instruments during their use. The End Effector 105B may be a handheld device or instrument used by the surgeon (e.g., a CORI® hand piece or a cutting guide or jig) or, alternatively, the End Effector 105B can include a device or instrument held or positioned by a robotic arm 105A. While one robotic arm 105A is illustrated in FIG. 1, in some embodiments there may be multiple devices. As examples, there may be one robotic arm 105A on each side of an operating table T or two devices on one side of the table T. The robotic arm 105A may be mounted directly to the table T, be located next to the table T on a floor platform (not shown), mounted on a floor-to-ceiling pole, or mounted on a wall or ceiling of an operating room. The floor platform may be fixed or moveable. In one particular embodiment, the robotic arm 105A is mounted on a floor-to-ceiling pole located between the patient's legs or feet. In some embodiments, the End Effector 105B may include a suture holder or a stapler to assist in closing wounds. Further, in the case of two robotic arms 105A, the surgical computer 150 can drive the robotic arms 105A to work together to suture the wound at closure. Alternatively, the surgical computer 150 can drive one or more robotic arms 105A to staple the wound at closure.

The Effector Platform 105 can include a Limb Positioner 105C for positioning the patient's limbs during surgery. One example of a Limb Positioner 105C is the SMITH AND NEPHEW SPIDER2 system. The Limb Positioner 105C may be operated manually by the surgeon or alternatively change limb positions based on instructions received from the Surgical Computer 150 (described below). While one Limb Positioner 105C is illustrated in FIG. 1, in some embodiments there may be multiple devices. As examples, there may be one Limb Positioner 105C on each side of the operating table T or two devices on one side of the table T. The Limb Positioner 105C may be mounted directly to the table T, be located next to the table T on a floor platform (not shown), mounted on a pole, or mounted on a wall or ceiling of an operating room. In some embodiments, the Limb Positioner 105C can be used in non-conventional ways, such as a retractor or specific bone holder. The Limb Positioner 105C may include, as examples, an ankle boot, a soft tissue clamp, a bone clamp, or a soft-tissue retractor spoon, such as a hooked, curved, or angled blade. In some embodiments, the Limb Positioner 105C may include a suture holder to assist in closing wounds.

The Effector Platform 105 may include tools, such as a screwdriver, light or laser, to indicate an axis or plane, bubble level, pin driver, pin puller, plane checker, pointer, finger, or some combination thereof.

Resection Equipment 110 (not shown in FIG. 1) performs bone or tissue resection using, for example, mechanical, ultrasonic, or laser techniques. Examples of Resection Equipment 110 include drilling devices, burring devices, oscillatory sawing devices, vibratory impaction devices, reamers, ultrasonic bone cutting devices, radio frequency ablation devices, reciprocating devices (such as a rasp or broach), and laser ablation systems. In some embodiments, the Resection Equipment 110 is held and operated by the surgeon during surgery. In other embodiments, the Effector Platform 105 may be used to hold the Resection Equipment 110 during use.

The Effector Platform 105 also can include a cutting guide or jig 105D that is used to guide saws or drills used to resect tissue during surgery. Such cutting guides 105D can be formed integrally as part of the Effector Platform 105 or robotic arm 105A or cutting guides can be separate structures that can be matingly and/or removably attached to the Effector Platform 105 or robotic arm 105A. The Effector Platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The Tracking System 115 uses one or more sensors to collect real-time position data that locates the patient's anatomy and surgical instruments. For example, for TKA procedures, the Tracking System may provide a location and orientation of the End Effector 105B during the procedure. In addition to positional data, data from the Tracking System 115 also can be used to infer velocity/acceleration of anatomy/instrumentation, which can be used for tool control. In some embodiments, the Tracking System 115 may use a tracker array attached to the End Effector 105B to determine the location and orientation of the End Effector 105B. The position of the End Effector 105B may be inferred based on the position and orientation of the Tracking System 115 and a known relationship in three-dimensional space between the Tracking System 115 and the End Effector 105B. Various types of tracking systems may be used in various embodiments of the present invention including, without limitation, Infrared (IR) tracking systems, electromagnetic (EM) tracking systems, video or image based tracking systems, and ultrasound registration and tracking systems. Using the data provided by the tracking system 115, the surgical computer 150 can detect objects and prevent collision. For example, the surgical computer 150 can prevent the robotic arm 105A and/or the End Effector 105B from colliding with soft tissue.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some embodiments, this can include stereoscopic, tri-scopic, quad-scopic, etc. imaging. In addition to the camera array, which in some embodiments is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system. In some embodiments, the camera may be mounted on the robotic arm 105A.

In some embodiments, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some embodiments, certain markers, such as fiducial marks that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one- or two-dimensional optical codes (barcode, QR code, etc.) can be affixed to objects in the theater to provide passive identification that can occur based on image analysis. If these codes are placed asymmetrically on an object, they also can be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some embodiments, augmented reality (AR) headsets can be worn by surgeons and other staff to provide additional camera angles and tracking capabilities. In this case, the infrared/time of flight sensor data, which is predominantly used for hand/gesture detection, can build correspondence between the AR headset and the tracking system of the robotic system using sensor fusion techniques. This can be used to calculate a calibration matrix that relates the optical camera coordinate frame to the fixed holographic world frame.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient also can involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process also can include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

A Tissue Navigation System 120 (not shown in FIG. 1) provides the surgeon with intraoperative, real-time visualization for the patient's bone, cartilage, muscle, nervous, and/or vascular tissues surrounding the surgical area. Examples of systems that may be employed for tissue navigation include fluorescent imaging systems and ultrasound systems.

The Display 125 provides graphical user interfaces (GUIs) that display images collected by the Tissue Navigation System 120 as well other information relevant to the surgery. For example, in one embodiment, the Display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The Display 125 may include, for example, one or more computer monitors. As an alternative or supplement to the Display 125, one or more members of the surgical staff may wear an Augmented Reality (AR) Head Mounted Device (HMD). For example, in FIG. 1 the Surgeon 111 is wearing an AR HMD 155 that may, for example, overlay pre-operative image data on the patient or provide surgical planning suggestions. In one embodiment, a tracker array-mounted surgical tool could be detected by both the IR camera and an AR headset (HMD) using sensor fusion techniques without the need for any “intermediate” calibration rigs. This near-depth, time-of-flight sensing camera located in the HMD could be used for hand/gesture detection. The headset's sensor API can be used to expose IR and depth image data and carryout image processing using, for example, C++ with OpenCV. This approach allows the relationship between the CASS and the virtual coordinate frame to be determined and the headset sensor data (i.e., IR in combination with depth images) to isolate the CASS tracker arrays. The image processing system on the HMD can locate the surgical tool in a fixed holographic world frame and the CASS IR camera can locate the surgical tool relative to its camera coordinate frame. This relationship can be used to calculate a calibration matrix that relates the CASS IR camera coordinate frame to the fixed holographic world frame. This means that if a calibration matrix has previously been calculated, the surgical tool no longer needs to be visible to the AR headset. However, a recalculation may be necessary if the CASS camera is accidentally moved in the workflow. Various example uses of the AR HMD 155 in surgical procedures are detailed in the sections that follow.

Surgical Computer 150 provides control instructions to various components of the CASS 100, collects data from those components, and provides general processing for various data needed during surgery. In some embodiments, the Surgical Computer 150 is a general-purpose computer. In other embodiments, the Surgical Computer 150 may be a parallel computing platform that uses multiple central processing units (CPUs) or graphics processing units (GPU) to perform processing. In some embodiments, the Surgical Computer 150 is connected to a remote server over one or more computer networks (e.g., the Internet). The remote server can be used, for example, for storage of data or execution of computationally intensive processing tasks.

Various techniques generally known in the art can be used for connecting the Surgical Computer 150 to the other components of the CASS 100. Moreover, the computers can connect to the Surgical Computer 150 using a mix of technologies. For example, the End Effector 105B may connect to the Surgical Computer 150 over a wired (i.e., serial) connection. The Tracking System 115, Tissue Navigation System 120, and Display 125 can similarly be connected to the Surgical Computer 150 using wired connections. Alternatively, the Tracking System 115, Tissue Navigation System 120, and Display 125 may connect to the Surgical Computer 150 using wireless technologies such as, without limitation, Wi-Fi, Bluetooth, Near Field Communication (NFC), or ZigBee.

### Robotic Arm

In some embodiments, the CASS 100 includes a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device) and have the ability to be locked in place (e.g., by a press of a button, voice activation, a surgeon removing a hand from the robotic arm, or other method).

In some embodiments, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B attached or integrated into a robotic arm 105A may include, without limitation, a burring device, a scalpel, a cutting device, a retractor, a joint tensioning device, or the like. In embodiments in which an end effector 105B is used, the end effector may be positioned at the end of the robotic arm 105A such that any motor control operations are performed within the robotic arm system. In embodiments in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the Surgical Computer 150.

In some embodiments, the robotic arm 105A can be moved by hand (e.g., by the surgeon) or with internal motors into its ideal position and orientation for the task being performed. In some embodiments, the robotic arm 105A may be enabled to operate in a “free” mode that allows the surgeon to position the arm into a desired position without being restricted. While in the free mode, the position and orientation of the robotic arm 105A may still be tracked as described above. In one embodiment, certain degrees of freedom can be selectively released upon input from user (e.g., surgeon) during specified portions of the surgical plan tracked by the Surgical Computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 also can place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other embodiments, movement of the robotic arm 105A or robotically controlled end effector 105B can be controlled entirely by the CASS 100 without any, or with only minimal, assistance or input from a surgeon or other medical professional. In still other embodiments, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

A robotic arm 105A may be used for holding the retractor. For example, in one embodiment, the robotic arm 105A may be moved into the desired position by the surgeon. At that point, the robotic arm 105A may lock into place. In some embodiments, the robotic arm 105A is provided with data regarding the patient's position, such that if the patient moves, the robotic arm can adjust the retractor position accordingly. In some embodiments, multiple robotic arms may be used, thereby allowing multiple retractors to be held or for more than one activity to be performed simultaneously (e.g., retractor holding & reaming).

The robotic arm 105A may also be used to help stabilize the surgeon's hand while making a femoral neck cut. In this application, control of the robotic arm 105A may impose certain restrictions to prevent soft tissue damage from occurring. For example, in one embodiment, the Surgical Computer 150 tracks the position of the robotic arm 105A as it operates. If the tracked location approaches an area where tissue damage is predicted, a command may be sent to the robotic arm 105A causing it to stop. Alternatively, where the robotic arm 105A is automatically controlled by the Surgical Computer 150, the Surgical Computer may ensure that the robotic arm is not provided with any instructions that cause it to enter areas where soft tissue damage is likely to occur. The Surgical Computer 150 may impose certain restrictions on the surgeon to prevent the surgeon from reaming too far into the medial wall of the acetabulum or reaming at an incorrect angle or orientation.

In some embodiments, the robotic arm 105A may be used to hold a cup impactor at a desired angle or orientation during cup impaction. When the final position has been achieved, the robotic arm 105A may prevent any further seating to prevent damage to the pelvis.

The surgeon may use the robotic arm 105A to position the broach handle at the desired position and allow the surgeon to impact the broach into the femoral canal at the desired orientation. In some embodiments, once the Surgical Computer 150 receives feedback that the broach is fully seated, the robotic arm 105A may restrict the handle to prevent further advancement of the broach.

The robotic arm 105A may also be used for resurfacing applications. For example, the robotic arm 105A may stabilize the surgeon while using traditional instrumentation and provide certain restrictions or limitations to allow for proper placement of implant components (e.g., guide wire placement, chamfer cutter, sleeve cutter, plan cutter, etc.). Where only a burr is employed, the robotic arm 105A may stabilize the surgeon's handpiece and may impose restrictions on the handpiece to prevent the surgeon from removing unintended bone in contravention of the surgical plan.

The robotic arm 105A may be a passive arm. As an example, the robotic arm 105A may be a CIRQ robot arm available from Brainlab AG. CIRQ is a registered trademark of Brainlab AG, Olof-Palme-Str. 9 81829, München, FED REP of GERMANY. In one particular embodiment, the robotic arm 105A is an intelligent holding arm as disclosed in U.S. Pat. No. 10,426,571 to Krinninger et al., U.S. Pat. No. 10,993,077 to Nowatschin et al., U.S. patent application Ser. No. 15/561,048 to Nowatschin et al., and U.S. Pat. No. 10,342,636 to Nowatschin et al., the entire contents of each of which is herein incorporated by reference.

### Surgical Procedure Data Generation and Collection

The various services that are provided by medical professionals to treat a clinical condition are collectively referred to as an “episode of care.” For a particular surgical intervention, the episode of care can include three phases: pre-operative, intra-operative, and post-operative. During each phase, data is collected or generated that can be used to analyze the episode of care in order to understand various features of the procedure and identify patterns that may be used, for example, in training models to make decisions with minimal human intervention. The data collected over the episode of care may be stored at the Surgical Computer 150 or the Surgical Data Server 180 as a complete dataset. Thus, for each episode of care, a dataset exists that comprises all of the data collectively pre-operatively about the patient, all of the data collected or stored by the CASS 100 intra-operatively, and any post-operative data provided by the patient or by a healthcare professional monitoring the patient.

As explained in further detail, the data collected during the episode of care may be used to enhance performance of the surgical procedure or to provide a holistic understanding of the surgical procedure and the patient outcomes. For example, in some embodiments, the data collected over the episode of care may be used to generate a surgical plan. In one embodiment, a high-level, pre-operative plan is refined intra-operatively as data is collected during surgery. In this way, the surgical plan can be viewed as dynamically changing in real-time or near real-time as new data is collected by the components of the CASS 100. In other embodiments, pre-operative images or other input data may be used to develop a robust plan preoperatively that is simply executed during surgery. In this case, the data collected by the CASS 100 during surgery may be used to make recommendations that ensure that the surgeon stays within the pre-operative surgical plan. For example, if the surgeon is unsure how to achieve a certain prescribed cut or implant alignment, the Surgical Computer 150 can be queried for a recommendation. In still other embodiments, the pre-operative and intra-operative planning approaches can be combined such that a robust pre-operative plan can be dynamically modified, as necessary or desired, during the surgical procedure. In some embodiments, a biomechanics-based model of patient anatomy contributes simulation data to be considered by the CASS 100 in developing preoperative, intraoperative, and post-operative/rehabilitation procedures to optimize implant performance outcomes for the patient.

Aside from changing the surgical procedure itself, the data gathered during the episode of care may be used as an input to other procedures ancillary to the surgery. For example, in some embodiments, implants can be designed using episode of care data. Example data-driven techniques for designing, sizing, and fitting implants are described in U.S. Pat. No. 10,064,686, filed Aug. 15, 2011, and entitled “Systems and Methods for Optimizing Parameters for Orthopaedic Procedures”; U.S. Pat. No. 10,102,309, filed Jul. 20, 2012 and entitled “Systems and Methods for Optimizing Fit of an Implant to Anatomy”; and U.S. Pat. No. 8,078,440, filed Sep. 19, 2008 and entitled “Operatively Tuning Implants for Increased Performance,” the entire contents of each of which are hereby incorporated by reference into this patent application.

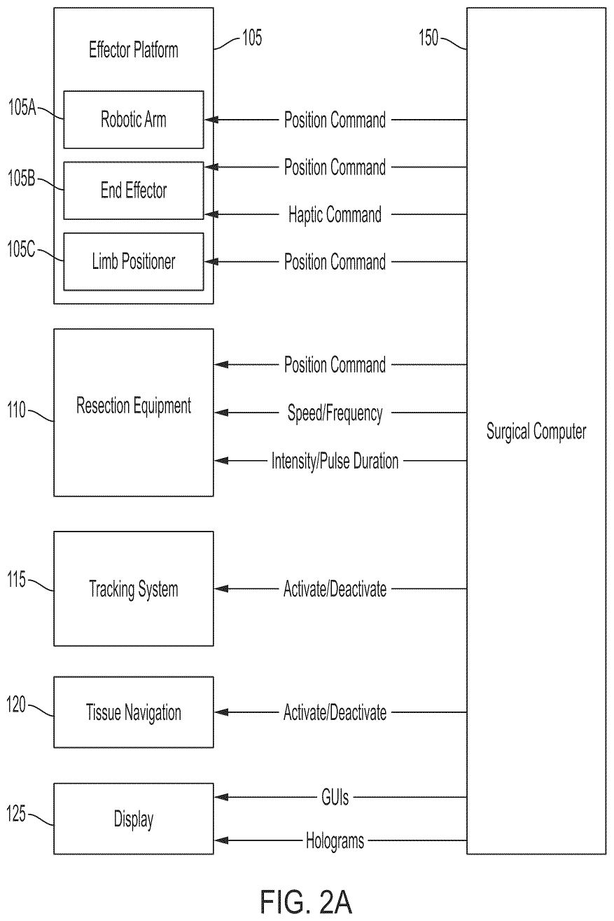

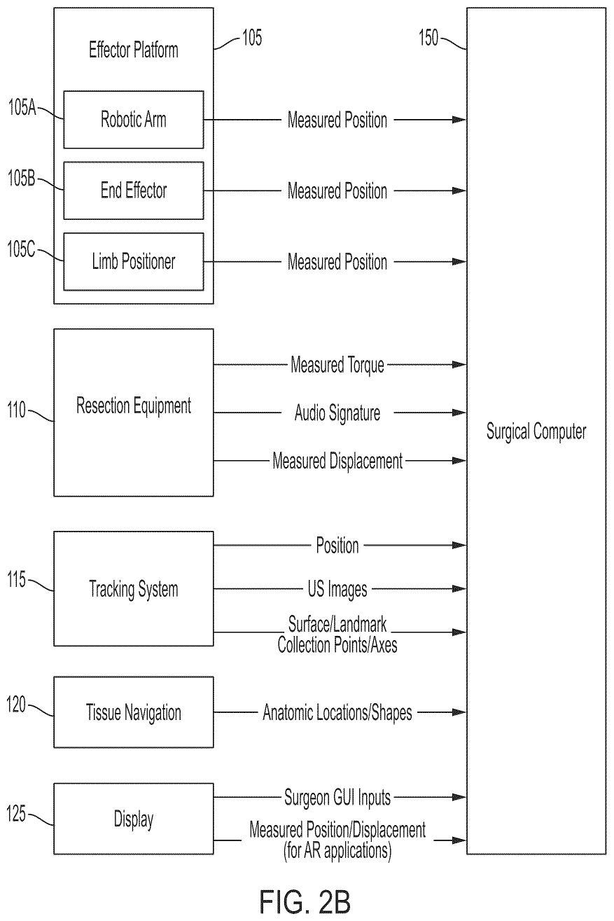

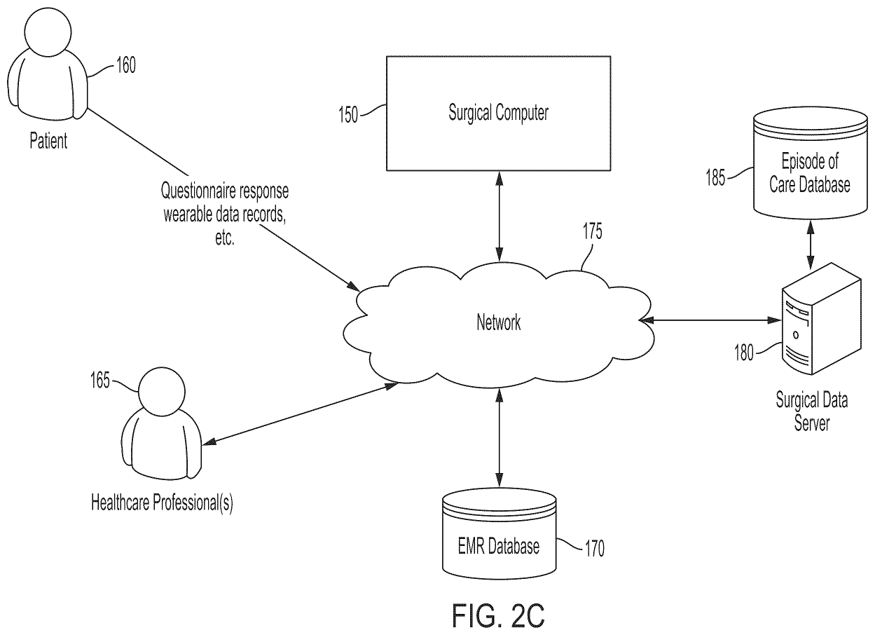

Furthermore, the data can be used for educational, training, or research purposes. For example, using the network-based approach described below in FIG. 2C, other doctors or students can remotely view surgeries in interfaces that allow them to selectively view data as it is collected from the various components of the CASS 100. After the surgical procedure, similar interfaces may be used to “playback” a surgery for training or other educational purposes, or to identify the source of any issues or complications with the procedure.

Data acquired during the pre-operative phase generally includes all information collected or generated prior to the surgery. Thus, for example, information about the patient may be acquired from a patient intake form or electronic medical record (EMR). Examples of patient information that may be collected include, without limitation, patient demographics, diagnoses, medical histories, progress notes, vital signs, medical history information, allergies, and lab results. The pre-operative data may also include images related to the anatomical area of interest. These images may be captured, for example, using Magnetic Resonance Imaging (MRI), Computed Tomography (CT), X-ray, ultrasound, or any other modality known in the art. The pre-operative data may also comprise quality of life data captured from the patient. For example, in one embodiment, pre-surgery patients use a mobile application (“app”) to answer questionnaires regarding their current quality of life. In some embodiments, preoperative data used by the CASS 100 includes demographic, anthropometric, cultural, or other specific traits about a patient that can coincide with activity levels and specific patient activities to customize the surgical plan to the patient. For example, certain cultures or demographics may be more likely to use a toilet that requires squatting on a daily basis.

FIGS. 2A and 2B provide examples of data that may be acquired during the intra-operative phase of an episode of care. These examples are based on the various components of the CASS 100 described above with reference to FIG. 1; however, it should be understood that other types of data may be used based on the types of equipment used during surgery and their use.

FIG. 2A shows examples of some of the control instructions that the Surgical Computer 150 provides to other components of the CASS 100, according to some embodiments. Note that the example of FIG. 2A assumes that the components of the Effector Platform 105 are each controlled directly by the Surgical Computer 150. In embodiments where a component is manually controlled by the Surgeon 111, instructions may be provided on the Display 125 or AR HMD 155 instructing the Surgeon 111 how to move the component.

The various components included in the Effector Platform 105 are controlled by the Surgical Computer 150 providing position commands that instruct the component where to move within a coordinate system. In some embodiments, the Surgical Computer 150 provides the Effector Platform 105 with instructions defining how to react when a component of the Effector Platform 105 deviates from a surgical plan. These commands are referenced in FIG. 2A as “haptic” commands. For example, the End Effector 105B may provide a force to resist movement outside of an area where resection is planned. Other commands that may be used by the Effector Platform 105 include vibration and audio cues.

In some embodiments, the end effectors 105B of the robotic arm 105A are operatively coupled with cutting guide 105D. In response to an anatomical model of the surgical scene, the robotic arm 105A can move the end effectors 105B and the cutting guide 105D into position to match the location of the femoral or tibial cut to be performed in accordance with the surgical plan. This can reduce the likelihood of error, allowing the vision system and a processor utilizing that vision system to implement the surgical plan to place a cutting guide 105D at the precise location and orientation relative to the tibia or femur to align a cutting slot of the cutting guide with the cut to be performed according to the surgical plan. Then, a surgeon can use any suitable tool, such as an oscillating or rotating saw or drill to perform the cut (or drill a hole) with perfect placement and orientation because the tool is mechanically limited by the features of the cutting guide 105D. In some embodiments, the cutting guide 105D may include one or more pin holes that are used by a surgeon to drill and screw or pin the cutting guide into place before performing a resection of the patient tissue using the cutting guide. This can free the robotic arm 105A or ensure that the cutting guide 105D is fully affixed without moving relative to the bone to be resected. For example, this procedure can be used to make the first distal cut of the femur during a total knee arthroplasty. In some embodiments, where the arthroplasty is a hip arthroplasty, cutting guide 105D can be fixed to the femoral head or the acetabulum for the respective hip arthroplasty resection. It should be understood that any arthroplasty that utilizes precise cuts can use the robotic arm 105A and/or cutting guide 105D in this manner.

The Resection Equipment 110 is provided with a variety of commands to perform bone or tissue operations. As with the Effector Platform 105, position information may be provided to the Resection Equipment 110 to specify where it should be located when performing resection. Other commands provided to the Resection Equipment 110 may be dependent on the type of resection equipment. For example, for a mechanical or ultrasonic resection tool, the commands may specify the speed and frequency of the tool. For Radiofrequency Ablation (RFA) and other laser ablation tools, the commands may specify intensity and pulse duration.

Some components of the CASS 100 do not need to be directly controlled by the Surgical Computer 150; rather, the Surgical Computer 150 only needs to activate the component, which then executes software locally specifying the manner in which to collect data and provide it to the Surgical Computer 150. In the example of FIG. 2A, there are two components that are operated in this manner: the Tracking System 115 and the Tissue Navigation System 120.

The Surgical Computer 150 provides the Display 125 with any visualization that is needed by the Surgeon 111 during surgery. For monitors, the Surgical Computer 150 may provide instructions for displaying images, GUIs, etc. using techniques known in the art. The display 125 can include various portions of the workflow of a surgical plan. During the registration process, for example, the display 125 can show a preoperatively constructed 3D bone model and depict the locations of the probe as the surgeon uses the probe to collect locations of anatomical landmarks on the patient. The display 125 can include information about the surgical target area. For example, in connection with a TKA, the display 125 can depict the mechanical and anatomical axes of the femur and tibia. The display 125 can depict varus and valgus angles for the knee joint based on a surgical plan, and the CASS 100 can depict how such angles will be affected if contemplated revisions to the surgical plan are made. Accordingly, the display 125 is an interactive interface that can dynamically update and display how changes to the surgical plan would impact the procedure and the final position and orientation of implants installed on bone.

As the workflow progresses to preparation of bone cuts or resections, the display 125 can depict the planned or recommended bone cuts before any cuts are performed. The surgeon 111 can manipulate the image display to provide different anatomical perspectives of the target area and can have the option to alter or revise the planned bone cuts based on intraoperative evaluation of the patient. The display 125 can depict how the chosen implants would be installed on the bone if the planned bone cuts are performed. If the surgeon 111 choses to change the previously planned bone cuts, the display 125 can depict how the revised bone cuts would change the position and orientation of the implant when installed on the bone.

The display 125 can provide the surgeon 111 with a variety of data and information about the patient, the planned surgical intervention, and the implants. Various patient-specific information can be displayed, including real-time data concerning the patient's health such as heart rate, blood pressure, etc. The display 125 also can include information about the anatomy of the surgical target region including the location of landmarks, the current state of the anatomy (e.g., whether any resections have been made, the depth and angles of planned and executed bone cuts), and future states of the anatomy as the surgical plan progresses. The display 125 also can provide or depict additional information about the surgical target region. For a TKA, the display 125 can provide information about the gaps (e.g., gap balancing) between the femur and tibia and how such gaps will change if the planned surgical plan is carried out. For a TKA, the display 125 can provide additional relevant information about the knee joint such as data about the joint's tension (e.g., ligament laxity) and information concerning rotation and alignment of the joint. The display 125 can depict how the planned implants' locations and positions will affect the patient as the knee joint is flexed. The display 125 can depict how the use of different implants or the use of different sizes of the same implant will affect the surgical plan and preview how such implants will be positioned on the bone. The CASS 100 can provide such information for each of the planned bone resections in a TKA or THA. In a TKA, the CASS 100 can provide robotic control for one or more of the planned bone resections. For example, the CASS 100 can provide robotic control only for the initial distal femur cut, and the surgeon 111 can manually perform other resections (anterior, posterior and chamfer cuts) using conventional means, such as a 4-in-1 cutting guide or jig 105D.

The display 125 can employ different colors to inform the surgeon of the status of the surgical plan. For example, un-resected bone can be displayed in a first color, resected bone can be displayed in a second color, and planned resections can be displayed in a third color. Implants can be superimposed onto the bone in the display 125, and implant colors can change or correspond to different types or sizes of implants.

The information and options depicted on the display 125 can vary depending on the type of surgical procedure being performed. Further, the surgeon 111 can request or select a particular surgical workflow display that matches or is consistent with his or her surgical plan preferences. For example, for a surgeon 111 who typically performs the tibial cuts before the femoral cuts in a TKA, the display 125 and associated workflow can be adapted to take this preference into account. The surgeon 111 also can preselect that certain steps be included or deleted from the standard surgical workflow display. For example, if a surgeon 111 uses resection measurements to finalize an implant plan but does not analyze ligament gap balancing when finalizing the implant plan, the surgical workflow display can be organized into modules, and the surgeon can select which modules to display and the order in which the modules are provided based on the surgeon's preferences or the circumstances of a particular surgery. Modules directed to ligament and gap balancing, for example, can include pre- and post-resection ligament/gap balancing, and the surgeon 111 can select which modules to include in their default surgical plan workflow depending on whether they perform such ligament and gap balancing before or after (or both) bone resections are performed.

For more specialized display equipment, such as AR HMDs, the Surgical Computer 150 may provide images, text, etc. using the data format supported by the equipment. For example, if the Display 125 is a holography device such as the Microsoft HoloLens™ or Magic Leap One™, the Surgical Computer 150 may use the HoloLens Application Program Interface (API) to send commands specifying the position and content of holograms displayed in the field of view of the Surgeon 111.

In some embodiments, one or more surgical planning models may be incorporated into the CASS 100 and used in the development of the surgical plans provided to the surgeon 111. The term “surgical planning model” refers to software that simulates the biomechanics performance of anatomy under various scenarios to determine the optimal way to perform cutting and other surgical activities. For example, for knee replacement surgeries, the surgical planning model can measure parameters for functional activities, such as deep knee bends, gait, etc., and select cut locations on the knee to optimize implant placement. One example of a surgical planning model is the LIFEMOD™ simulation software from SMITH AND NEPHEW, INC. In some embodiments, the Surgical Computer 150 includes computing architecture that allows full execution of the surgical planning model during surgery (e.g., a GPU-based parallel processing environment). In other embodiments, the Surgical Computer 150 may be connected over a network to a remote computer that allows such execution, such as a Surgical Data Server 180 (see FIG. 2C). As an alternative to full execution of the surgical planning model, in some embodiments, a set of transfer functions are derived that simplify the mathematical operations captured by the model into one or more predictor equations. Then, rather than execute the full simulation during surgery, the predictor equations are used. Further details on the use of transfer functions are described in WIPO Publication No. 2020/037308, filed Aug. 19, 2019, entitled “Patient Specific Surgical Method and System,” the entirety of which is incorporated herein by reference.

FIG. 2B shows examples of some of the types of data that can be provided to the Surgical Computer 150 from the various components of the CASS 100. In some embodiments, the components may stream data to the Surgical Computer 150 in real-time or near real-time during surgery. In other embodiments, the components may queue data and send it to the Surgical Computer 150 at set intervals (e.g., every second). Data may be communicated using any format known in the art. Thus, in some embodiments, the components all transmit data to the Surgical Computer 150 in a common format. In other embodiments, each component may use a different data format, and the Surgical Computer 150 is configured with one or more software applications that enable translation of the data.

In general, the Surgical Computer 150 may serve as the central point where CASS data is collected. The exact content of the data will vary depending on the source. For example, each component of the Effector Platform 105 provides a measured position to the Surgical Computer 150. Thus, by comparing the measured position to a position originally specified by the Surgical Computer 150 (see FIG. 2B), the Surgical Computer can identify deviations that take place during surgery.

The Resection Equipment 110 can send various types of data to the Surgical Computer 150 depending on the type of equipment used. Example data types that may be sent include the measured torque, audio signatures, and measured displacement values. Similarly, the Tracking Technology 115 can provide different types of data depending on the tracking methodology employed. Example tracking data types include position values for tracked items (e.g., anatomy, tools, etc.), ultrasound images, and surface or landmark collection points or axes. The Tissue Navigation System 120 provides the Surgical Computer 150 with anatomic locations, shapes, etc. as the system operates.

Although the Display 125 generally is used for outputting data for presentation to the user, it may also provide data to the Surgical Computer 150. For example, for embodiments where a monitor is used as part of the Display 125, the Surgeon 111 may interact with a GUI to provide inputs which are sent to the Surgical Computer 150 for further processing. For AR applications, the measured position and displacement of the HMD may be sent to the Surgical Computer 150 so that it can update the presented view as needed.

During the post-operative phase of the episode of care, various types of data can be collected to quantify the overall improvement or deterioration in the patient's condition as a result of the surgery. The data can take the form of, for example, self-reported information reported by patients via questionnaires. For example, in the context of a knee replacement surgery, functional status can be measured with an Oxford Knee Score questionnaire, and the post-operative quality of life can be measured with a EQ5D-5L questionnaire. Other examples in the context of a hip replacement surgery may include the Oxford Hip Score, Harris Hip Score, and WOMAC (Western Ontario and McMaster Universities Osteoarthritis index). Such questionnaires can be administered, for example, by a healthcare professional directly in a clinical setting or using a mobile app that allows the patient to respond to questions directly. In some embodiments, the patient may be outfitted with one or more wearable devices that collect data relevant to the surgery. For example, following a knee surgery, the patient may be outfitted with a knee brace that includes sensors that monitor knee positioning, flexibility, etc. This information can be collected and transferred to the patient's mobile device for review by the surgeon to evaluate the outcome of the surgery and address any issues. In some embodiments, one or more cameras can capture and record the motion of a patient's body segments during specified activities postoperatively. This motion capture can be compared to a biomechanics model to better understand the functionality of the patient's joints and better predict progress in recovery and identify any possible revisions that may be needed.

The post-operative stage of the episode of care can continue over the entire life of a patient. For example, in some embodiments, the Surgical Computer 150 or other components comprising the CASS 100 can continue to receive and collect data relevant to a surgical procedure after the procedure has been performed. This data may include, for example, images, answers to questions, “normal” patient data (e.g., blood type, blood pressure, conditions, medications, etc.), biometric data (e.g., gait, etc.), and objective and subjective data about specific issues (e.g., knee or hip joint pain). This data may be explicitly provided to the Surgical Computer 150 or other CASS component by the patient or the patient's physician(s). Alternatively, or additionally, the Surgical Computer 150 or other CASS component can monitor the patient's EMR and retrieve relevant information as it becomes available. This longitudinal view of the patient's recovery allows the Surgical Computer 150 or other CASS component to provide a more objective analysis of the patient's outcome to measure and track success or lack of success for a given procedure. For example, a condition experienced by a patient long after the surgical procedure can be linked back to the surgery through a regression analysis of various data items collected during the episode of care. This analysis can be further enhanced by performing the analysis on groups of patients that had similar procedures and/or have similar anatomies.

In some embodiments, data is collected at a central location to provide for easier analysis and use. Data can be manually collected from various CASS components in some instances. For example, a portable storage device (e.g., USB stick) can be attached to the Surgical Computer 150 into order to retrieve data collected during surgery. The data can then be transferred, for example, via a desktop computer to the centralized storage. Alternatively, in some embodiments, the Surgical Computer 150 is connected directly to the centralized storage via a Network 175 as shown in FIG. 2C.

FIG. 2C illustrates a “cloud-based” implementation in which the Surgical Computer 150 is connected to a Surgical Data Server 180 via a Network 175. This Network 175 may be, for example, a private intranet or the Internet. In addition to the data from the Surgical Computer 150, other sources can transfer relevant data to the Surgical Data Server 180. The example of FIG. 2C shows three additional data sources: the Patient 160, Healthcare Professional(s) 165, and an EMR Database 170. Thus, the Patient 160 can send pre-operative and post-operative data to the Surgical Data Server 180, for example, using a mobile app. The Healthcare Professional(s) 165 includes the surgeon and his or her staff as well as any other professionals working with Patient 160 (e.g., a personal physician, a rehabilitation specialist, etc.). It should also be noted that the EMR Database 170 may be used for both pre-operative and post-operative data. For example, assuming that the Patient 160 has given adequate permissions, the Surgical Data Server 180 may collect the EMR of the Patient pre-surgery. Then, the Surgical Data Server 180 may continue to monitor the EMR for any updates post-surgery.

At the Surgical Data Server 180, an Episode of Care Database 185 is used to store the various data collected over a patient's episode of care. The Episode of Care Database 185 may be implemented using any technique known in the art. For example, in some embodiments, a SQL-based database may be used where all of the various data items are structured in a manner that allows them to be readily incorporated in two SQL's collection of rows and columns. However, in other embodiments a No-SQL database may be employed to allow for unstructured data, while providing the ability to rapidly process and respond to queries. As is understood in the art, the term “No-SQL” is used to define a class of data stores that are non-relational in their design. Various types of No-SQL databases may generally be grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein and, in some embodiments, the different types of databases may support the Episode of Care Database 185.

Data can be transferred between the various data sources and the Surgical Data Server 180 using any data format and transfer technique known in the art. It should be noted that the architecture shown in FIG. 2C allows transmission from the data source to the Surgical Data Server 180, as well as retrieval of data from the Surgical Data Server 180 by the data sources. For example, as explained in detail below, in some embodiments, the Surgical Computer 150 may use data from past surgeries, machine learning models, etc. to help guide the surgical procedure.

In some embodiments, the Surgical Computer 150 or the Surgical Data Server 180 may execute a de-identification process to ensure that data stored in the Episode of Care Database 185 meets Health Insurance Portability and Accountability Act (HIPAA) standards or other requirements mandated by law. HIPAA provides a list of certain identifiers that must be removed from data during de-identification. The aforementioned de-identification process can scan for these identifiers in data that is transferred to the Episode of Care Database 185 for storage. For example, in one embodiment, the Surgical Computer 150 executes the de-identification process just prior to initiating transfer of a particular data item or set of data items to the Surgical Data Server 180. In some embodiments, a unique identifier is assigned to data from a particular episode of care to allow for re-identification of the data if necessary.

Although FIGS. 2A-C discuss data collection in the context of a single episode of care, it should be understood that the general concept can be extended to data collection from multiple episodes of care. For example, surgical data may be collected over an entire episode of care each time a surgery is performed with the CASS 100 and stored at the Surgical Computer 150 or at the Surgical Data Server 180. As explained in further detail below, a robust database of episode of care data allows the generation of optimized values, measurements, distances, or other parameters and other recommendations related to the surgical procedure. In some embodiments, the various datasets are indexed in the database or other storage medium in a manner that allows for rapid retrieval of relevant information during the surgical procedure. For example, in one embodiment, a patient-centric set of indices may be used so that data pertaining to a particular patient or a set of patients similar to a particular patient can be readily extracted. This concept can be similarly applied to surgeons, implant characteristics, CASS component versions, etc.

Further details of the management of episode of care data are described in U.S. Pat. No. 11,532,402 to Farley et al., the entirety of which is incorporated herein by reference.

### Extended Reality Registration

The present disclosure describes illustrative systems and methods of tracking specific portions of a patient's skeletal structure during surgery using a surgical navigation system enhanced by an extended reality system. Extended reality, as disclosed herein, refers to immersive learning technologies including virtual reality, augmented reality, and mixed reality.

The disclosed tracking system is particularly adapted for surgical procedures that utilize surgical navigation systems, such as the CORI® surgical navigation system. Such procedures can include knee replacement surgery, hip replacement surgery, shoulder arthroplasty, revision surgery or the like.

Robotic surgical navigation typically relies on a tracking system to track patient anatomy and tools during a surgical procedure. In some embodiments, tracking elements are attached rigidly to instruments and/or screwed or clamped to one or more bones using bone fixation hardware. Such tracking systems are often based on optical sensors (e.g., an infrared camera system that monitors the relative position of one or more reflective markers attached to the frames within its line of sight). It should be noted that although the bulk of the disclosure is directed toward optical tracking, that is merely for illustrative purposes and that, in other embodiments, the tracking system's sensor may be adapted to sense an electrical signal, a magnetic field, an electromagnetic field, a sound, a physical body, radio frequency, an x-ray, light, an active signal, a passive signal, and/or the like.

The designs of these systems are typically very specific to the constraints of the operating room. Moreover, the frames are configured for simplified sterilization, often made of steel or aluminum, and are designed to be large enough to be viewed from one or more cameras mounted within the operating room.

During surgical navigation, a surgeon is typically guided by a graphical user interface (GUI), which is displayed on one or more monitors in the operating room. These monitors may be mounted on a cart, a bed, or the like. Because the monitors are heavy and may require various wired connections, their general locations relative to the patient and/or surgeon are typically fixed. Because of this, surgeons are often required to look away or turn their head to view the information presented on the display. As a result, the surgeon's attention can be diverted from the surgical site.

In order to simplify the process for surgeons, various embodiments are discussed herein related to using augmented reality displays as a way to bring a GUI directly into the line of sight of the surgeon. Such embodiments may include projecting a virtual screen wherever the surgeon is looking (e.g., directly over the surgical site). Using augmented reality, the GUI may be projected on a virtual display in or adjacent to the surgeon's line of site during surgery, and thus, the surgeon does not have to turn from the surgical site while operating.

In some further embodiments, an augmented reality system may project surgical data (e.g., surgical plan information) directly onto the patient's anatomy (e.g., for an orthopedic surgery, the patient's bone may be visualized). A primary issue with using augmented reality for surgical procedures is ensuring that the data being shown is exact regarding both location and information. In order for augmented reality systems to project surgical data so that it properly aligns with a bone or other anatomy, the system calculates how to display the information relative to the patient's body such that it aligns with the surgeon's view of the scene (e.g., through a near-eye display, a virtual projection, or a display device).

Current “video-see-through” augmented reality systems are able to do this by tracking markers rigidly affixed to objects in a scene. However, these systems employ a video camera rigidly mounted to an augmented reality display, and this assembly (i.e., the display and camera) is then placed between a user and the scene. When the user looks at the display, the display shows the scene captured by the camera as well as the overlaid data in a single view. Therefore, the video image may present an image very similar to what the user would see if the user was looking “through” the display.

To place virtual objects into a scene, video-see-through augmented reality systems may superimpose graphical renderings of various virtual objects into a video image. However, if the virtual objects are to be perceived as part of the actual scene, they must be anchored to the scene by tracking real objects in the actual scene. Video-based augmented reality systems generally use markers that are recognizable by some form of image processing software. The video frames are then analyzed by an algorithm that identifies one or more markers and calculates how the marker should be oriented and positioned relative to the camera's location based on how it appears in the video image. Using these markers, an augmented reality system can track objects within the purview of the video tracking system.

Accordingly, in some embodiments, if at least one bone is being tracked and a surgical plan is known, the surgical augmented reality system may display the surgical plan superimposed directly on the view of the bone relative to the tracking markers' position on the bone as opposed to an abstraction of the plan displayed on a virtual GUI screen.

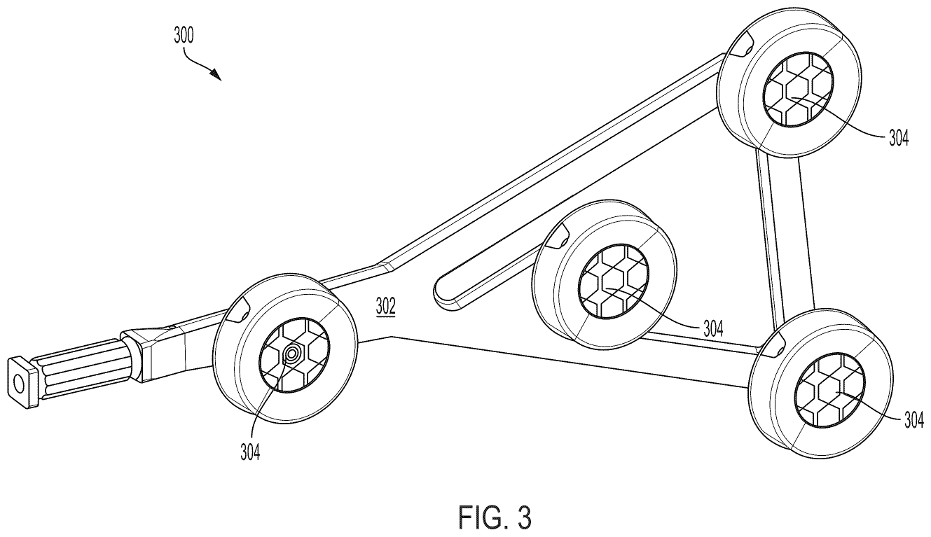

Referring to FIG. 3, an illustrative embodiment of a tracking device 300 is shown. As should be understood by those skilled in the art, various shapes and orientations of tracking devices may be used in computer-assisted surgical procedures based on the type of surgery and the surgical location. Thus, although FIG. 3 shows an optical tracking device with a specific configuration, it should be understood that this is merely illustrative and that any form of patient and/or bone tracking device may be used.

In some embodiments, and as shown in FIG. 3, a tracking device 300 may have multiple tracking elements 304. The tracking elements 304 may be mounted to a tracking frame 302. Because the elements 304 are detected by one or more sensor devices (e.g., tracking system 115), the tracking elements 304 may be at different locations and oriented at different angles on the tracking device 300 to ensure the best possible viewing angle between the tracking device 300 and the sensor (not shown). In a further embodiment, each tracking device 300, regardless of the number of tracking elements 304, may have an associated tracking reference frame (i.e., spatial orientation) associated with it. Thus, the CASS 100 is able to determine the exact location and orientation of the tracking device 300 based on the known tracking reference frame. The tracking elements 304 may include fiducial markers configured for use with an optical tracking system, for example. The use of optical tracking devices, such as the tracking device shown in FIG. 3, are well known in the art, and thus one of ordinary skill in the art would understand the purpose of the plurality of tracking elements 304 as well as the use of one or more sensors (not shown) to detect a patient, patient bone location, instrument, or other object within the surgical space. Further, additional details regarding tracking systems, including optical tracking systems, can be found above in relation to the tracking system 115 shown in FIGS. 2A and 2B.

Using the same marker/frame for both an augmented reality system and an optical navigation tracking system may be impractical for multiple reasons. For example, the tracking markers used in current augmented reality systems are generally very different from the tracking markers employed by surgical navigation systems. In addition, the performance requirements of a surgical navigation system tracking (e.g., accuracy or response time) are likely to be much higher than a typical augmented reality system. As a result, a surgical navigation system with augmented reality may require enhanced processing as compared to general video-based augmented reality markers.

Generally, existing surgical navigation systems that try to merge multiple tracking modalities often use a reference object that has a rigid configuration of a frame/marker to co-register a tracking system's coordinate frames. In most systems, dual-modality marker/frames are impractical due to the different design constraints present in different systems. For example, augmented reality markers (e.g., those used in ARToolKit) require high contrast black-and-white markers thatare typically made of paper or plastic and are produced using laser printing. However, typical navigational tracker frames (e.g., those used in the CORI® system) are usually metal and have reflective tracking markers. Thus, the markers used in typical navigational tracker systems may not have the high contrast needed for robust video tracking using augmented reality trackers. Furthermore, laser markings on sterilizable tools typically fade or otherwise deteriorate over time due to the sanitization process.

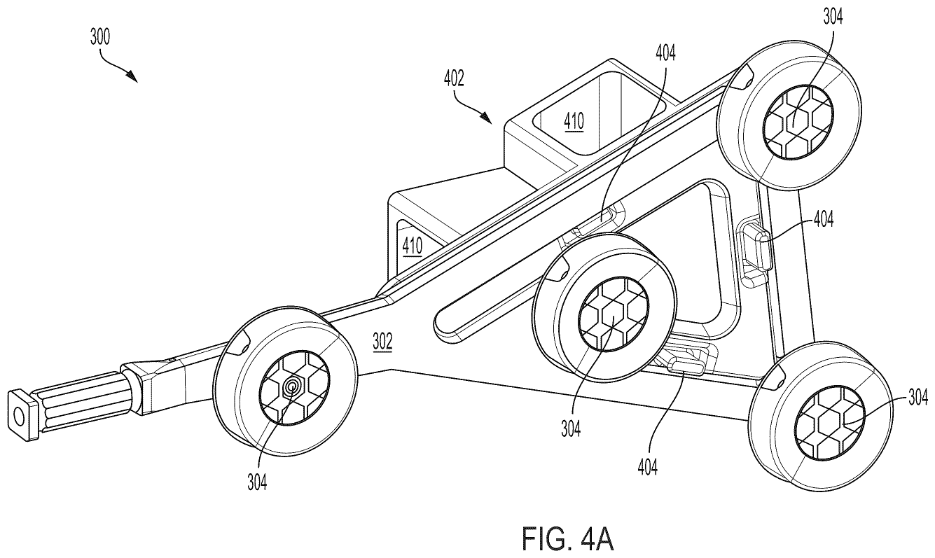

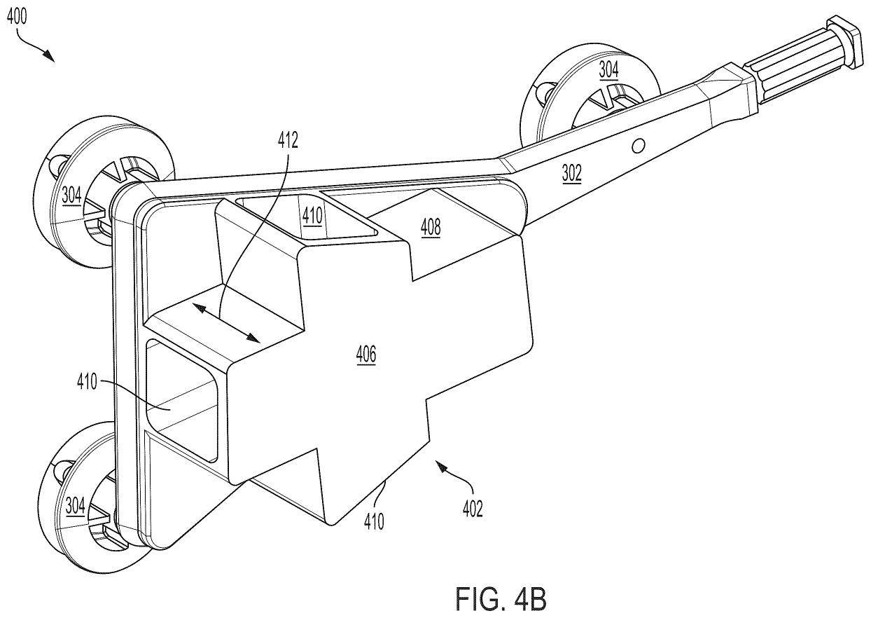

Accordingly, various embodiments are disclosed herein related to a video-augmented reality-compatible marker integrated with a tracking device 300 that may already be in use for computer-assisted surgery. In some embodiments, as shown in FIGS. 4A and 4B, an integrated marker assembly 400 may include an augmented reality marker assembly 402 attached to a tracking device 300 in a known position and orientation for co-registering the surgical navigational and augmented reality tracking modalities. The augmented reality-compatible marker may be removably interfaced to the tracking device 300.

In some embodiments, the augmented reality marker assembly 402 includes a recognizable three-dimensional shape with sufficient depth 412 and features to reliably register the augmented reality tracking system. A sufficient depth 412 may vary based on the accuracy requirements of the augmented reality tracking system. The features may include distinctive surface shapes 406/408 and openings 410 to a fully or partially hollow center. The features may further include distinctive (e.g., contrasting) colors or patterns applied to a portion of the surfaces 406/408/410. In the example illustrated in FIGS. 4A and 4B, the augmented reality system may detect, in a captured image, portions of the image corresponding to some combination of a lead surface 406, an edge surface 408, and an opening 410. Based on the detected geometries and the known geometry of the augmented reality marker assembly 402, the augmented reality system may determine a position and orientation of the augmented reality marker assembly 402.

In some embodiments, an augmented reality system may use a plurality of augmented reality marker assemblies 402. For example, one or more augmented reality marker assemblies 402 may track patient anatomy while an additional augmented reality marker assembly 402 tracks a surgical device. The augmented reality system may be configured to distinguish between the plurality of augmented reality marker assemblies 402. Each augmented reality marker assembly 402 may be distinguished based on a unique combination of shape 406/408/410, colors, or patterns. In further embodiments, a different symbol (e.g., a QR code) may be applied to one or more surfaces of each augmented reality marker assembly 402 to identify the specific assembly.

In certain embodiments, the augmented reality marker assembly 402 may be made of injection-molded plastic and have a distinctive shape, color, and/or a laser marking, which allows for the proper high-contrast needed for an augmented reality tracking system. In alternative embodiments, the augmented reality marker assembly 402 may be printed by a three-dimensional printer. An augmented reality marker assembly 402 constructed of plastic may be sterilized as a single-use component. Moreover, because augmented reality marker assemblies 402 may be produced relatively inexpensively, they can still provide a sterile environment and support a disposable usage model. In a further embodiment, augmented reality marker assemblies 402 may be custom-made for any kind of new or existing reusable tracking device (e.g., those currently used in computer-assisted surgery tracking).

In certain embodiments, in order to utilize two separate tracking systems (e.g., optical tracking devices 300 in combination with augmented reality marker assemblies 402), a transformation may be determined between the two reference frames. The transformation may include a matrix that transforms between the two spaces. A reliable transformation between the augmented reality reference frame and surgical navigation reference frame may allow data from the surgical navigation system to be displayed accurately and in real-time in augmented reality system.

In one or more embodiments, in order to utilize two separate tracking systems (e.g., optical tracking devices 300 in combination with augmented reality marker assemblies 402), the reference frames (i.e., coordinate systems) for each system need to be known. Thus, in some embodiments, a pre-calibrated reference object may be used to perform a co-registration process. Thus, in some embodiments, a pre-calibration step may be performed by identifying common points in each coordinate reference frame (e.g., by collecting fiduciary landmarks with a data collection probe or point probe, as described above under the heading “Registration of Pre-operative Data to Patient Anatomy using the Point Probe”) in each modality. By way of non-limiting example, a point probe, which may resemble a metal rod, may have its tip inserted or placed at one or more specific points on each tracker to orient and locate the tracker relative to each other and/or a stored coordinate of a know position in the computer system. Although this calibration step may be carried out individually for each tracker, in some embodiments, the landmarks can be collected simultaneously using a probe with tracking objects associated with each type of tracking modality.

In certain embodiments, the augmented reality marker(s) 402 may be manufactured such that their three-dimensional orientation is known relative to the three-dimensional tracking reference frame of the standard tracking device 300 when attached to the tracking device 300, such as is shown in FIGS. 4A and 4B. In other embodiments, one or more augmented reality marker assemblies 402 may be utilized in conjunction with the tracking device 300 to coordinate the reference frames for the separate tracking systems. Thus, in some embodiments, the augmented reality marker assembly 402 may be designed such that it can incorporate fastening mechanisms 404 that are configured to snap onto or otherwise connect to a known existing tracker device 300 (e.g., one of the various trackers used with the CORI® system) in a unique way. In other words, the fastening mechanisms 404 may be designed such that the augmented reality marker assembly 402 are affixed to the tracking device 1100 in a particular known or fixed orientation. The fastening mechanisms 404 may include semi-rigid clips configured to bias against the frame 302.

In some embodiments, the spatial relationship between the geometry of the augmented reality marker assembly 402 and the geometry of the tracking device(s) 300 is predetermined. As discussed herein, because the augmented reality marker assembly 402 can be made via injection molding and laser printing, a low error tolerance for each marker 402 may be repeatably and reliably manufactured with high accuracy to ensure a precise fit with the existing tracking device(s) 300.

Thus, in a further embodiment, an existing computer-assisted surgery system would be able to support an augmented reality display (e.g., near-eye display, video display, projection, etc.) without requiring substantial changes to the design of the various instruments used with the existing system. By way of example, in one embodiment, an existing system may simply broadcast surgical plan data that has been collected and analyzed with an associated tracking reference frame (i.e., three-dimensional coordinate system) of the existing tracking device 300 to an augmented reality system. The augmented reality system, which could identify/determine the co-registration between the existing CASS 100 and the augmented reality marker assembly 402, may then convert the surgical plan data to its own coordinate system or a coordinate system associated with the marker assembly 402 for display.

Accordingly, various embodiments are disclosed herein that comprise a tracking device 300 comprising one or more tracking points elements 304 and that is configured to be mounted to a patient before or during a surgical procedure. The one or more tracking elements 304 may be detected by a sensor or group of sensors capable of identifying the location of the one or more tracking elements 304, such as a camera of the tracking system 115 described in connection with FIGS. 2A and 2B. The sensor(s) may be connected to a processor (e.g., associated with the surgical computer 150) executing a computer program that captures the three-dimensional location of the tracking device 300. In some embodiments, the sensor(s) may also include an image capture device which detects and identifies the orientation of an augmented reality marker assembly 402. In additional or alternative embodiments, an additional image capture device (e.g., cameras located on a near-eye augmented reality device or body cameras) may be used to capture the augmented reality marker assembly 402.

Because, as discussed herein, the three-dimensional tracking reference frame of the tracking device 300 is known relative to the three-dimensional orientation of the augmented reality marker assembly 402, the CASS 100 may identify a relationship between the reference frames. In a further embodiment, the CASS 100 may cause an augmented reality device to display surgical data (e.g., information associated with a surgical plan) in a video-see-through view according to the determined relationship between the three-dimensional tracking reference frame of the tracking device 300 and the three-dimensional orientation of the augmented reality marker assembly 402.

In certain embodiments, the augmented reality marker assembly 402 may include one or more augmented reality symbols (e.g., a QR code or two-dimensional image). The one or more augmented reality symbols may include information identifying each of the plurality of augmented reality markers 402. Thus, the system may not only determine which augmented reality marker assembly 402 is attached, but also whether the marker is the correct type of marker for the type of surgery being performed (e.g., according to the surgical plan data).

In additional embodiments, the marker assembly 402 may be designed such that the symbol faces the user's augmented reality device when attached to a tracking frame 302. This is beneficial because a surgeon may not face in the direction of the one or more sensor devices (e.g., navigation system cameras) during the surgical procedure. Various embodiments may allow for different designs associated with different surgical approaches and/or operating room layouts. As discussed herein, the specific design and use case of each augmented reality marker assembly 402 may be denoted by a unique signature code embedded in the one or more augmented reality symbols.

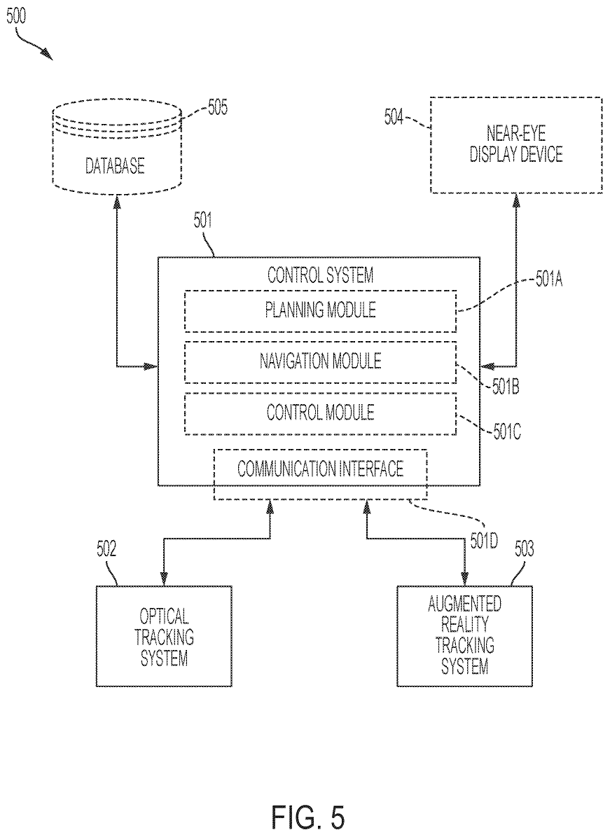

FIG. 5 is a block diagram depicting an illustrative system 500 for providing navigation and control to a computer-assisted surgical planning system. In an embodiment, the system 500 may include a control system 501, a tracking system 502, and an augmented reality tracking system 503. The control system 501 may include or be incorporated into the CASS 100. For example, the control system 501 may include the surgical computer 150 described in connection with FIGS. 1-2C. The tracking system 502 may include the tracking system 115 described above in connection with FIGS. 2A and 2B. The system 500 may also include a display device (e.g., a near-eye display device 504, a head-mounted display, or the display 125) and a database 505. In an example, these components may be combined to provide navigation and guidance during an orthopedic (or similar) prosthetic implant surgery.

The control system 501 may include one or more computing devices configured to coordinate information received from the tracking systems 502 and 503 and provide augmented reality in a video-see-through format to the near-eye display device 504. In an example, the control system 501 may include a planning module 501A, a navigation module 501B, a control module 501C, and a communication interface 501D. The planning module 501A may provide pre-operative planning services that enable clinicians to virtually plan a procedure prior to entering the operating room. Various methods of pre-operative planning are well known in the art. One specific example may be found in U.S. Pat. No. 6,205,411 titled “Computer-Assisted Surgery Planner and Intra-Operative Guidance System,” which is incorporated herein by reference in its entirety.

In one non-limiting example, the planning module 501A may be used to manipulate a virtual model of an implant in reference to a virtual implant host model and display it in a video-see-through format to the display device 504. The implant host model may be constructed from scans of the target patient. Such scans may include computed tomography (CT), magnetic resonance imaging (MRI), positron emission tomographic (PET), or ultrasound scans of the joint and surrounding structure. Alternatively, pre-operative planning may be performed by selecting a predefined implant host model from a group of models based on patient measurements or other clinician-selected inputs. In certain examples, pre-operative planning is refined intra-operatively by measuring the patient's (i.e., target implant host's) actual anatomy. In an example, a point probe, discussed herein connected to the tracking systems 502 and 503, may be used to measure the target implant host's actual anatomy and the relative locations of various tracking devices (e.g., 300, 400). Further, the planning module 501A may be configured to generate a surgical plan, such as is described above under the heading “Surgical Procedure Data Generation and Collection,” for example.

In an embodiment, the navigation module 501B may coordinate tracking the location and orientation of the tracking devices 300/400 relative to an implant or implant host. In certain examples, the navigation module 501B may also coordinate tracking of the virtual models used during pre-operative planning within the planning module 501A. Tracking the virtual models may include operations such as alignment of the virtual models with the implant host through data obtained via the tracking systems 502 and 503. In an embodiment, the navigation module 501B receives input from the tracking systems 502 and 503 regarding the physical location and orientation of the patient and the patient's specific anatomy. Tracking of the implant host may include tracking multiple individual bone structures. For example, during a total knee replacement procedure, the tracking system 502 and 503 may individually track the femur and/or the tibia using tracking devices (e.g., 300, 400) anchored to the individual bones.

In an embodiment, the control module 501C may process information provided by the navigation module 501B to generate control signals for controlling the view shown in the near-eye display device 504. In certain examples, the control module 501C may also work with the navigation module 501B to produce visual animations to assist the surgeon during an operative procedure. Visual animations may be displayed via a display device 504. In an embodiment, the visual animations may include a real-time 3-D representation of a patient's anatomy and/or an implant, among other things (e.g., information related to the surgical plan). In certain examples, the visual animations are color-coded to further assist the surgeon with positioning and orientation of the implant.

In an embodiment, the communication interface 501D facilitates communication between the control system 501 and external systems and devices. The communication interface 501D may include both wired and wireless communication interfaces, such as Ethernet, IEEE 802.11 wireless, or Bluetooth, among others. In such an embodiment, the primary external systems connected via the communication interface 501D include the tracking systems 502 and 503. Although not shown, the database 505 and the display device 504, among other devices, may also be connected to the control system 501 via the communication interface 501D. In an embodiment, the communication interface 501D communicates over an internal bus to other modules and hardware systems within the control system 501.

In an embodiment, the tracking systems 502 and 503 provide location and orientation information for surgical devices and trackers as they relate to each other to assist in navigation and control of semi-active robotic surgical devices. The tracking systems 502 and 503 may include a tracking device 300 that includes or otherwise provides tracking data based on at least three positions and at least three angles as well as an augmented reality tracking device 402. The tracking device 300 may include one or more first tracking markers associated with the implant host, and one or more second markers associated with a surgical device. The markers or some of the markers may be one or more of optical sources (e.g., infrared of visual range), Radio Frequency (RF) sources, ultrasound sources, and/or transmitters. The tracking systems 502 and 503 may thus include infrared tracking systems, optical tracking systems, ultrasound tracking systems, inertial tracking systems, wired systems, and/or RF tracking systems. One illustrative tracking system is the OPTOTRAK® 3-D motion and position measurement and tracking system, although those of ordinary skill in the art will recognize that other tracking systems of other accuracies and/or resolutions can be used.

Imagery for detecting the augmented reality marker assembly 402 can be captured using an imaging sensor associated with the AR HMD 155, the Tracking System 115, and/or any other camera (e.g., a mobile device or a dedicated camera).

Although the examples herein refer to applications in augmented reality, one of ordinary skill in the art will understand how they could be adapted to apply to any extended reality (XR) system.

### Data Processing Systems for Implementing Embodiments Herein

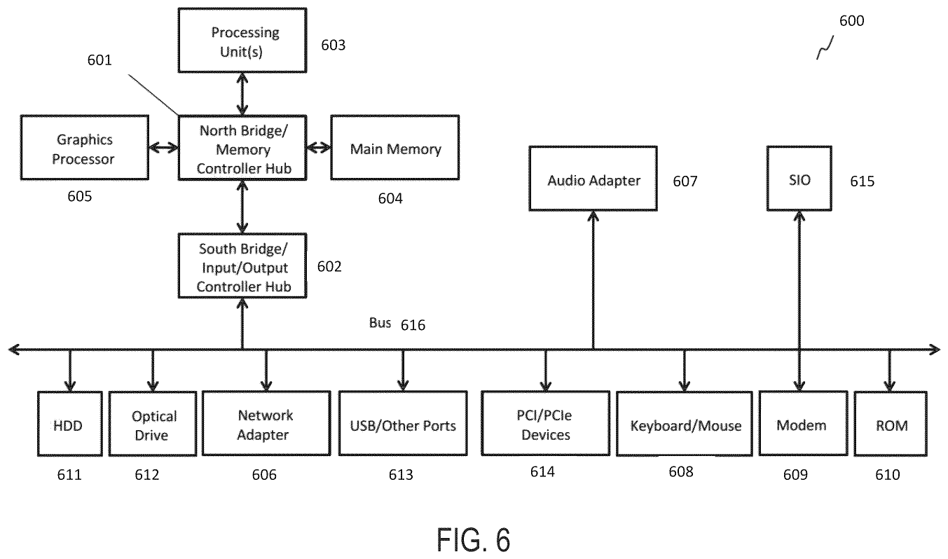

FIG. 6 illustrates a block diagram of an exemplary data processing system 600 in which embodiments are implemented. The data processing system 600 is an example of a computer, such as a server or client, in which computer usable code or instructions implementing the process for illustrative embodiments of the present invention are located. In some embodiments, the data processing system 600 may be a server computing device. For example, data processing system 600 can be implemented in a server or another similar computing device operably connected to a surgical system 100 or control system 501 as described above. The data processing system 600 can be configured to, for example, transmit and receive information related to a patient and/or a related surgical plan with the surgical system 100.

In the depicted example, data processing system 600 can employ a hub architecture including a north bridge and memory controller hub (NB/MCH) 601 and south bridge and input/output (I/O) controller hub (SB/ICH) 602. Processing unit 603, main memory 604, and graphics processor 605 can be connected to the NB/MCH 601. Graphics processor 605 can be connected to the NB/MCH 601 through, for example, an accelerated graphics port (AGP).

In the depicted example, a network adapter 606 connects to the SB/ICH 602. An audio adapter 607, keyboard and mouse adapter 608, modem 609, read only memory (ROM) 610, hard disk drive (HDD) 611, optical drive (e.g., CD or DVD) 612, universal serial bus (USB) ports and other communication ports 613, and PCI/PCIe devices 614 may connect to the SB/ICH 602 through bus system 616. PCI/PCIe devices 614 may include Ethernet adapters, add-in cards, and PC cards for notebook computers. ROM 610 may be, for example, a flash basic input/output system (BIOS). The HDD 611 and optical drive 612 can use an integrated drive electronics (IDE) or serial advanced technology attachment (SATA) interface. A super I/O (SIO) device 615 can be connected to the SB/ICH 602.

An operating system can run on the processing unit 603. The operating system can coordinate and provide control of various components within the data processing system 600. As a client, the operating system can be a commercially available operating system. An object-oriented programming system, such as the Java™ programming system, may run in conjunction with the operating system and provide calls to the operating system from the object-oriented programs or applications executing on the data processing system 600. As a server, the data processing system 600 can be an IBM® eServer™ System® running the Advanced Interactive Executive operating system or the Linux operating system. The data processing system 600 can be a symmetric multiprocessor (SMP) system that can include a plurality of processors in the processing unit 603. Alternatively, a single processor system may be employed.

Instructions for the operating system, the object-oriented programming system, and applications or programs are located on storage devices, such as the HDD 611, and are loaded into the main memory 604 for execution by the processing unit 603. The processes for embodiments described herein can be performed by the processing unit 603 using computer usable program code, which can be located in a memory such as, for example, main memory 604, ROM 610, or in one or more peripheral devices.

A bus system 616 can be comprised of one or more busses. The bus system 616 can be implemented using any type of communication fabric or architecture that can provide for a transfer of data between different components or devices attached to the fabric or architecture. A communication unit such as the modem 609 or the network adapter 606 can include one or more devices that can be used to transmit and receive data.

Those of ordinary skill in the art will appreciate that the hardware depicted in FIG. 6 may vary depending on the implementation. Other internal hardware or peripheral devices, such as flash memory, equivalent non-volatile memory, or optical disk drives may be used in addition to or in place of the hardware depicted. Moreover, the data processing system 600 can take the form of any of a number of different data processing systems, including but not limited to, client computing devices, server computing devices, tablet computers, laptop computers, telephone or other communication devices, personal digital assistants, and the like. Essentially, data processing system 600 can be any known or later developed data processing system without architectural limitation.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices also can “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., +10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A surgical system comprising:
a tracking device comprising a first tracking marker configured for use with a surgical navigation tracking system, the tracking device configured to be mounted to a patient;
an augmented reality marker assembly comprising a second tracking marker configured for use with an augmented reality system, the augmented reality marker assembly configured to be attached to the tracking device at a fixed orientation, wherein the second tracking marker comprises a contrasting three-dimensional surface;
a sensor assembly configured to identify locations of each of the first tracking marker and the second tracking marker; and
a control system communicably connected to the sensor assembly, the control system configured to:
determine a first three-dimensional orientation of the patient in a first reference frame by locating the first tracking marker via the sensor assembly, and
determine a second three-dimensional orientation of the augmented reality marker assembly in a second reference frame by locating the second tracking marker via the sensor assembly.

2. The surgical system of claim 1, wherein the control system is further configured to determine a transformation between the first reference frame and the second reference frame.

3. The surgical system of claim 2, wherein the control system comprises a display screen, wherein the control system is further configured to cause the display screen to display surgical data based on the transformation.

4. The surgical system of claim 3, wherein the display screen comprises at least one of a near-eye display or a head-mounted display.

5. The surgical system of claim 3, wherein:
the control system is further configured to generate a surgical plan; and
at least a portion of the surgical data displayed via the display screen comprises the surgical plan.

6. The surgical system of claim 1, wherein the contrasting three-dimensional surface comprises a plurality of contrasting colors.

7. The surgical system of claim 6, wherein the contrasting three-dimensional surface further comprises a plurality of polygonal surfaces, wherein a color of each polygonal surface is selected from the plurality of contrasting colors.

8. The surgical system of claim 1, wherein the contrasting three-dimensional surface comprises one or more openings into a hollow portion of the second tracking marker.

9. The surgical system of claim 1, wherein:
the augmented reality marker assembly comprises a plurality of fastening mechanisms; and
the plurality of fastening mechanisms are configured to be attached to a frame of the tracking device in a particular manner that causes the augmented reality marker assembly to assume the fixed orientation.

10. The surgical system of claim 1, further comprising:
a plurality of tracking devices; and
a plurality of augmented reality marker assemblies each comprising an augmented reality tracking marker;
wherein each augmented reality tracking marker is distinguishable by the sensor assembly.

11. The surgical system of claim 1, wherein the sensor assembly comprises:
a first sensor configured to identify a first location of the first tracking marker; and
a second sensor configured to identify a second location of the second tracking marker.

12. The surgical system of claim 1, wherein the sensor assembly comprises a plurality of tracking cameras.

13. A tracking device attachment for correlating a tracking system coordinate frame with an augmented reality system coordinate frame, the tracking device attachment comprising:
a tracking marker comprising a contrasting three-dimensional surface configured to be identified by an augmented reality system; and
a plurality of fastening mechanisms configured to attach the tracking device attachment to a tracking device at a fixed orientation, wherein the tracking device comprises a marker configured to be identified by a surgical navigation system and configured to be mounted to a patient.

14. The tracking device attachment of claim 13, wherein the plurality of fastening mechanisms are configured to be attached to a frame of the tracking device in a particular manner that causes the tracking device attachment to assume the fixed orientation.

15. The tracking device attachment of claim 13, wherein the contrasting three-dimensional surface comprises a plurality of contrasting colors.

16. The tracking device attachment of claim 15, wherein the contrasting three-dimensional surface further comprises a plurality of polygonal surfaces, wherein a color of each polygonal surface is selected from the plurality of contrasting colors.

17. The tracking device attachment of claim 15, wherein the plurality of contrasting colors form a pattern on the contrasting three-dimensional surface.

18. The tracking device attachment of claim 13, wherein the contrasting three-dimensional surface comprises one or more openings into a hollow portion of the tracking marker.

19. A surgical system comprising:
a sensor assembly configured to:
identify a first location of a first tracking marker associated with a tracking device, the tracking device configured to be mounted to a patient, wherein the first tracking marker is configured for use with a surgical navigation system, and
identify a second location of a second tracking marker associated with an augmented reality marker assembly, the augmented reality marker assembly configured to be attached to the tracking device at a fixed orientation, wherein the second tracking marker comprises a contrasting three-dimensional surface configured for use with an augmented reality system; and
a control system communicably connected to the sensor assembly, the control system configured to:
determine a first three-dimensional orientation, in a first reference frame of the patient by locating the first tracking marker via the sensor assembly, and
determine a second three-dimensional orientation, in a second reference frame, of the augmented reality marker assembly by locating the second tracking marker via the sensor assembly.

20. The surgical system of claim 19, wherein the control system is further configured to determine a transformation between the first reference frame and the second reference frame.
